(19) United States
(12) Patent Application Publication
(10) Pub. No.: US 2026/0102605 A1
(43) Pub. Date: Apr. 16, 2026

(54) LVAD-SUPPORT UNDERGARMENT

(71) Applicants: Timothy Caito, Monongahela, PA (US); Maria Greer, Monongahela, PA (US)

(72) Inventors: Timothy Caito, Monongahela, PA (US); Maria Greer, Monongahela, PA (US)

(21) Appl. No.: 18/916,043

(22) Filed: Oct. 15, 2024

(51) Int. Cl. A61M 60/90

(52) U.S. Cl. CPC A61M 60/90; A61M 2209/088

(57) ABSTRACT

A LVAD-support undergarment includes underpants, a plurality of pockets attached to the underpants and designed to securely hold external LVAD components, and a securing strap attached to the underpants and designed to hold a LVAD wire close to a body of a wearer.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to medical support undergarments and more particularly pertains to a new support undergarment for holding components of a left ventricular assist device (LVAD). The LVAD is a device for people suffering heart failure. The LVAD uses a mechanical pump that is implanted in the chest cavity and connected to the heart. The pump helps the lower left chamber, or left ventricle, of the heart pump blood out of the ventricle to the aorta and then to the rest of the body. The LVAD has several components, specifically the pump, a controller, a driveline connecting the pump to the controller, and a power supply. The driveline is a wire or cable that passes from the pump through an opening in the torso of person to the controller, which is a small computer that controls the operation of the LVAD. The controller runs the pump and provides messages and alarms regarding operation of the LVAD. The power supply is usually a pair of rechargeable batteries that are connected by wires to the controller. These components are usually worn by the person using an external shoulder harness or backpack. The drawbacks of the harness or backpack design is that the LVAD is highly visible, which can attract unwanted attention, and the components and wires are exposed to accidental or inadvertent contact that could damage the LVAD or affect the operation thereof. For example, the driveline could become snagged on an object and accidentally disconnected, which could be at best inconvenient and at worst dangerous to the user. The new LVAD-support undergarment disclosed herein provides a different type of support for the LVAD that is wearable under normal clothes. This concealed design prevents unwanted attention as others cannot see the LVAD. In addition, the LVAD-support undergarment includes one or more securing straps that hold the different wires close to the body to minimize accidental snags and disconnections.

### (2) Description of Related Art Including Information Disclosed Under 37 CFR 1.97 and 1.98

The prior art relates to medical support undergarments, such as LVAD harnesses and backpacks. The prior art, as best understood, does not disclose a LVAD-support undergarment that includes underpants, a plurality of pockets attached to the underpants and designed to securely hold external LVAD components, and a securing strap attached to the underpants and designed to hold a LVAD wire close to a body of a wearer.

### BRIEF SUMMARY OF THE INVENTION

An embodiment of the disclosure meets the needs presented above in a LVAD-support undergarment generally comprising underpants, a plurality of pockets attached to the underpants and designed to securely hold external LVAD components, and a securing strap attached to the underpants and designed to hold a LVAD wire close to a body of a wearer.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a LVAD-support undergarment according to an embodiment of the disclosure.

FIG. 2 is a rear view of the embodiment of the disclosure shown in FIG. 1.

FIG. 3 is a front view of the embodiment of the disclosure shown in FIG. 1 in use.

FIG. 4 is a front view of an embodiment of the disclosure in use.

FIG. 5 is a front view of an embodiment of the disclosure.

FIG. 6 is a rear view of the embodiment of the disclosure shown in FIG. 5.

FIG. 7 is a front view of an embodiment of the disclosure in use.

### DETAILED DESCRIPTION OF THE INVENTION

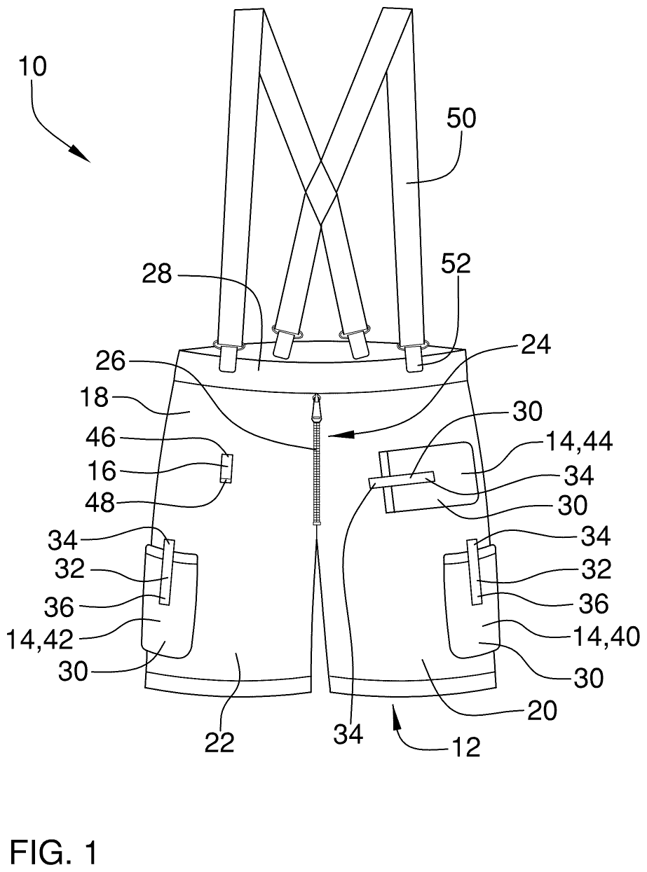

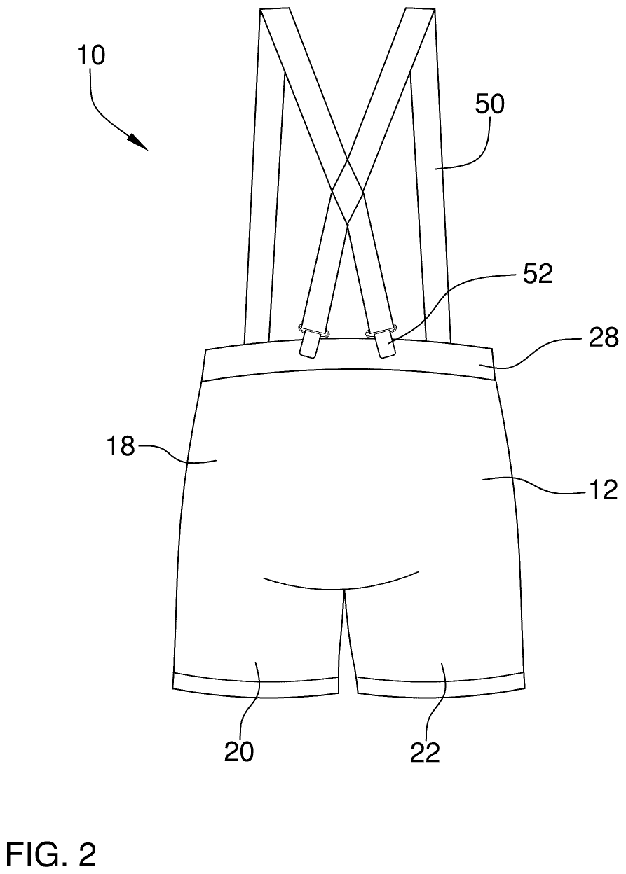

With reference now to the drawings, and in particular to FIGS. 1 through 7 thereof, a new LVAD-support undergarment embodying the principles and concepts of an embodiment of the disclosure and generally designated by the reference numeral 10 will be described.

As best illustrated in FIGS. 1 through 7, the LVAD-support undergarment 10 generally comprises underpants 12, a plurality of pockets 14 attached to the underpants 12 and designed to securely hold external LVAD components, and a securing strap 16 attached to the underpants 12 and designed to hold a LVAD wire close to a body of a wearer.

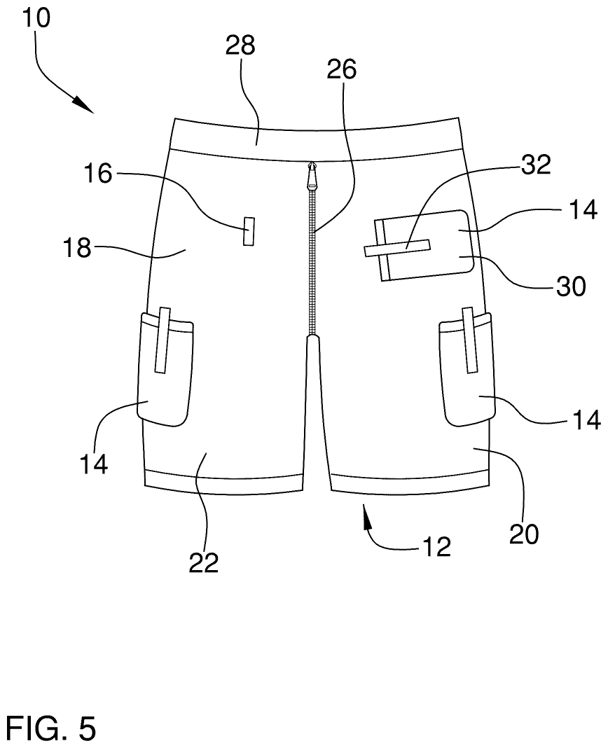

In the figures, FIGS. 1 through 4 show an embodiment of the LVAD-support undergarment 10 designed for men, and FIGS. 5 through 7 show an embodiment of the LVAD-support undergarment 10 designed for women, though components of each could be used in one or more possible embodiments. For clarity, some reference numbers have been omitted for identical elements in the embodiments.

The underpants 12 could be most any type of common underpants 12, such as long underpants, midway briefs, boxer shorts, boxer briefs, and trunk briefs. Other types of underpants 12, such as briefs, shapewear, or bikini briefs, could possibly be adapted for use in the LVAD-support undergarment 10. In the exemplary embodiment shown in FIGS. 1 through 4, the underpants 12 are boxer shorts. In the exemplary embodiment shown in FIGS. 5 through 7, the underpants 12 are boxer briefs. These types of underpants 12 include a pelvic section 18, a first leg section 20, and a second leg section 22.

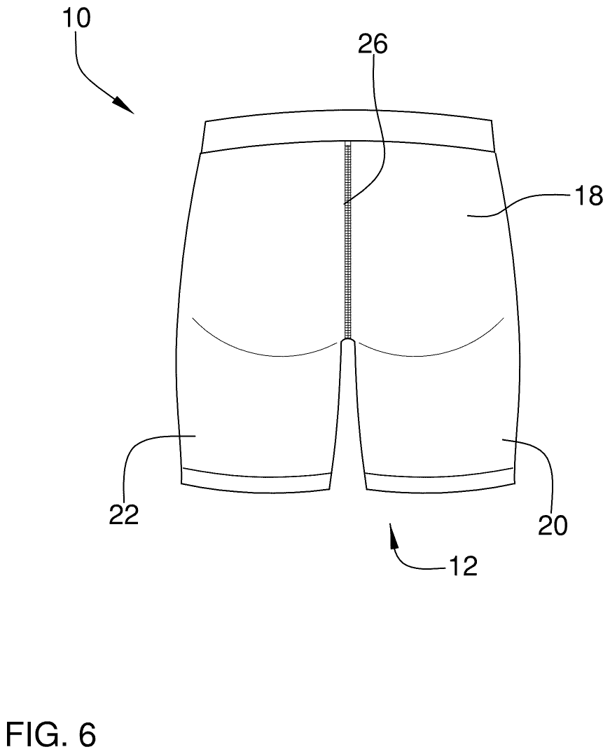

The underpants 12 could include components common to different types of underpants 12. In the exemplary embodiment, the pelvic section 18 includes a fly 24 that is openable and closable, which fly 24 includes a zipper 26. With reference to FIGS. 5 and 6, the zipper 26 could be designed to extend between the first and second leg sections 20, 22 from front to back of the pelvic section 18. This design is advantageous for women, as it would allow the wearer to urinate without having to remove the underpants 12. Such a design could also be useful for men and women as the user could possibly defecate without having to remove the underpants 12. The underpants 12 also include a waist band 28. The waist band 28 could include resilient elastic material. In addition, in one possible embodiment, the waist band 28 could include a mechanical securing structure, such as a drawstring, a button, or a snap, either in place of or in combination with the resilient elastic material, as is well known in the clothing arts.

Each of the pockets 14 includes a receptacle 30 and a cover element 32 releasably attached to the receptacle 30 to secure LVAD components in the receptacle 30. In the exemplary embodiment shown in the figures, the cover element 32 is strap-like and has a first cover end 34 attached to the underpants 12 and a second cover end 36 releasably attached to the receptacle 30. The cover element 32 could have other designs, such as a flap. In the exemplary embodiment shown in the figures, the second cover end 36 is releasably attached by hook and loop material 38, though other connecting structures, such as snaps, buttons, or zippers are within the scope of the disclosure. The receptacle 30 and the cover element 32 could be made from fabric or cloth that is the same as or different than the material from which the underpants 12 are made. The receptacle 30 and the cover element 32 could also include or be made of resiliently stretchable elastic material to be able to accommodate LVAD components of different shapes and sizes.

In the exemplary embodiment shown, the pockets 14 include a first pocket 40 designed to hold a first LVAD battery 60, a second pocket 42 designed to hold a second LVAD battery 62, and a third pocket 44 designed to hold a LVAD controller. More pockets 14 could be included for additional components as needed. In the exemplary embodiment, the first pocket 40 is positioned at a left side of the underpants 12 and the second pocket 42 is positioned at a right side of the underpants 12, in this case on the first leg section 20 and the second leg section 22, respectively. The third pocket 44 is positioned on the front of the pelvic section 18 adjacent the first leg section 20, though it could be positioned elsewhere in another possible embodiment, such as adjacent the second leg section 22 or on the back of the pelvic section 18. Any of the pockets 14 could be placed on the sides of the underpants 12, that is, adjacent the hips or the sides of the legs of a wearer. The pockets 14 could possibly be located at the buttocks or the backs of the legs of the wearer. In the exemplary embodiment, the third pocket 44 is oriented horizontally, while each of the first pocket 40 and the second pocket 42 is oriented vertically. However, the pockets 14 could be oriented in essentially any direction that best suits the connection of the LVAD components.

The securing strap 16 is used to hold down the LVAD wires or cables, such as the driveline 66 or a battery wire 70. In the exemplary embodiment shown, the securing strap 16 includes a first strap end 46 attached to the underpants 12 and a second strap end 48 releasably attached to the underpants 12. In one possible embodiment, the securing strap 16 could be a loop wherein both ends are attached to the underpants 12 and the LVAD wires could be threaded through the loop. The securing strap 16 could also include resiliently stretchable material. Like the pockets 14, the securing strap 16 could be made from fabric or cloth that is the same as or different than the material from which the underpants 12 are made. Like the cover element 32, the second strap end 48 can be releasably attached by hook and loop material, though other connecting structures, such as snaps, buttons, or zippers are within the scope of the disclosure.

In the exemplary embodiment shown in FIGS. 1 through 4, the LVAD-support undergarment 10 further includes suspenders 50 connected to the underpants 12. The suspenders 50 are detachably connected to the underpants 12 by clasps 52, though in one possible embodiment, the suspenders 50 could be permanently affixed by adhesive or stitching. While the suspenders 50 are shown in conjunction with the embodiment of the underpants 12 for men, the suspenders 50 could also be used with the embodiment of the underpants 12 for women.

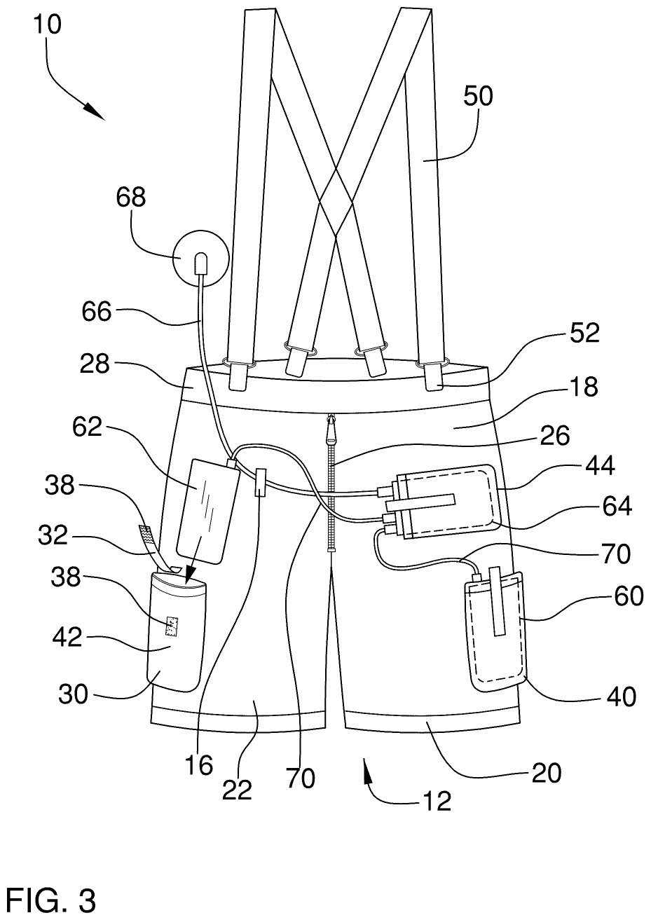

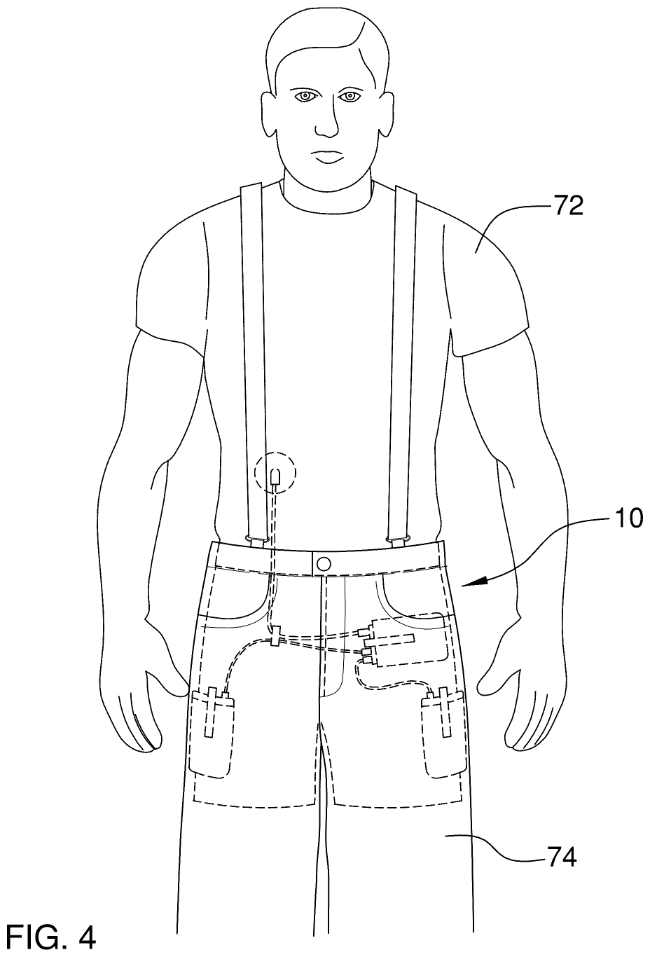

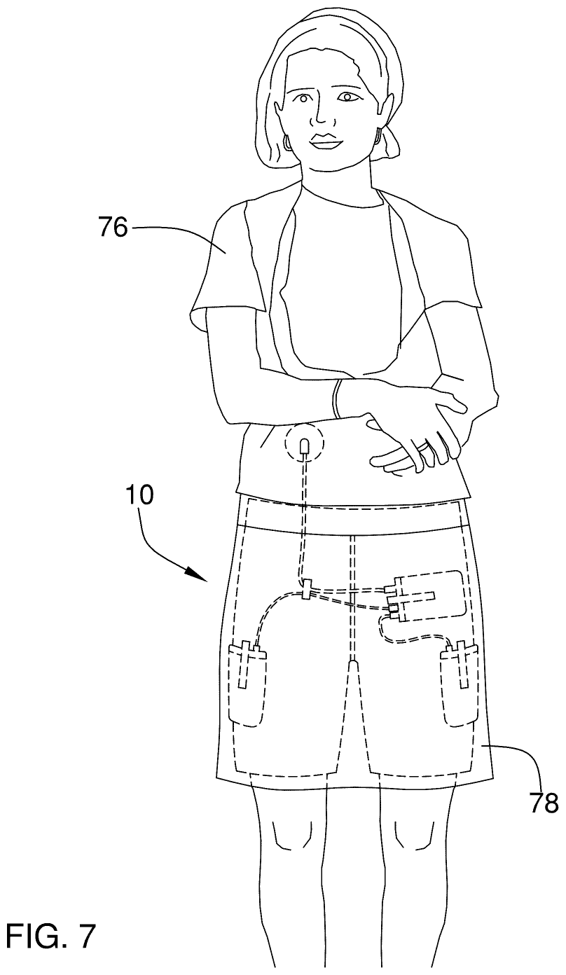

FIG. 3 shows how the LVAD components fit into the LVAD-support undergarment 10. The first LVAD battery 60 is inserted into the first pocket 40 and the second LVAD battery 62 into the second pocket 42. The controller 64 is inserted into the third pocket 44. The cover element 32 secures these LVAD components in the pockets 14. The driveline 66, which runs from the controller 64 and into a portal 68, is held against the underpants 12 by the securing strap 16. As shown in FIGS. 4 and 7, the battery wire 70 from the second LVAD battery 62 is also held by the securing strap 16. As shown in FIG. 4, the wearer, in this case a man 72, can wear pants 74 over the LVAD-support undergarment 10. The suspenders 50 help hold up the underpants 12. As shown in FIG. 7, the wearer, in this case a woman 76, is wearing the LVAD-support undergarment 10 under a skirt 78. The suspenders 50 are not used in this embodiment, as the underpants 12 are of a form-fitting design and material, such as a stretchable resilient elastic material.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A LVAD-support undergarment comprising:
underpants;
a plurality of pockets attached to said underpants and configured to securely hold external LVAD components; and
a securing strap attached to said underpants and configured to hold a LVAD wire close to a body of a wearer.

2. The LVAD-support undergarment of claim 1, wherein said underpants comprise one of: long underpants, midway briefs, boxer shorts, boxer briefs, and trunk briefs.

3. The LVAD-support undergarment of claim 2, wherein said underpants comprise a pelvic section, a first leg section, and a second leg section.

4. The LVAD-support undergarment of claim 3, wherein said pelvic section comprises a fly that is openable and closable.

5. The LVAD-support undergarment of claim 4, wherein said fly comprises a zipper.

6. The LVAD-support undergarment of claim 5, wherein said zipper extends between said first and second leg sections from front to back of said pelvic section.

7. The LVAD-support undergarment of claim 1, wherein said underpants comprise a waist band that comprises resilient elastic material.

8. The LVAD-support undergarment of claim 1, wherein said pockets comprise a receptacle and a cover element releasably attached to said receptacle to secure LVAD components in said receptacle.

9. The LVAD-support undergarment of claim 8, wherein said cover element has a first cover end attached to said underpants and a second cover end releasably attached to said receptacle.

10. The LVAD-support undergarment of claim 1, wherein said pockets comprise a first pocket configured to hold a first LVAD battery, a second pocket configured to hold a second LVAD battery, and a third pocket configured to hold a controller.

11. The LVAD-support undergarment of claim 10, wherein said first pocket is disposed at a left side of said underpants, and said second pocket is disposed at a right side of said underpants.

12. The LVAD-support undergarment of claim 1, wherein said securing strap comprises a first strap end attached to said underpants and a second strap end releasably attached to said underpants.

13. The LVAD-support undergarment of claim 1, wherein said securing strap comprises resiliently stretchable material.

14. The LVAD-support undergarment of claim 1, wherein the LVAD-support undergarment further comprises suspenders connected to said underpants.

15. The LVAD-support undergarment of claim 14, wherein said suspenders are detachably connected to said underpants by clasps.

16. The LVAD-support undergarment of claim 1, wherein.
said underpants comprise one of: long underpants, midway briefs, boxer shorts, boxer briefs, and trunk briefs;
said underpants comprise a pelvic section, a first leg section, and a second leg section;
said pelvic section comprises a fly that is openable and closable, wherein:
said fly comprises a zipper;
said zipper extends between said first and second leg sections from front to back of said pelvic section;
said underpants comprise a waist band that comprises resilient elastic material;
said pockets comprise a receptacle and a cover element releasably attached to said receptacle to secure LVAD components in said receptacle;
said cover element has a first cover end attached to said underpants and a second cover end releasably attached to said receptacle;
said pockets comprise a first pocket configured to hold a first LVAD battery, a second pocket configured to hold a second LVAD battery, and a third pocket configured to hold a controller, wherein said first pocket is disposed at a left side of said underpants and said second pocket is disposed at a right side of said underpants;
said securing strap comprises a first strap end attached to said underpants and a second strap end releasably attached to said underpants; and
said securing strap comprises resiliently stretchable material.

17. The LVAD-support undergarment of claim 16, wherein the LVAD-support undergarment further comprises suspenders connected to said underpants, and said suspenders are detachably connected to said underpants by clasps.
